(19) United States
(12) Patent Application Publication
(10) Pub. No.: US 2024/0081635 A1
(43) Pub. Date: Mar. 14, 2024

(54) REFRACTIVE TEST CARD AND MEASUREMENT METHOD THEREFOR

(71) Applicants: Chun Ho LAU, Shanghai (CN); Chun Put LAU, Hong Kong (CN)

(72) Inventors: Chun Ho LAU, Shanghai (CN); Chun Put LAU, Hong Kong (CN)

(21) Appl. No.: 18/273,673

(22) PCT Filed: Mar. 4, 2022
(86) PCT No.: PCT/CN2022/079250

§ 371 Date: Jul. 21, 2023

(30) Foreign Application Priority Data
Jan. 21, 2021 (CN) 2021100839153

(51) Int. Cl. A61B 3/032; A61B 3/036

(52) U.S. Cl. CPC A61B 3/032; A61B 3/036

(57) ABSTRACT

A set of refractive test cards (30, 40, 50) and measurement methods therefor are provided. The test cards (30, 40, 50) involves optotypes (32, 42, 52) under a black background (31, 41, 51). The optotypes (32, 42, 52) comprises a partition unit (33, 43, 53). The partition unit (33, 43, 53) visually separates the optotypes (32, 42, 52). The methods comprises: determining whether a subject has astigmatism based on sharpness of contrast between optotypes (32, 42) and a black background (31, 41); in response to determining that the subject has astigmatism, determining an astigmatic axis; determining whether the subject has myopia or hyperopia based on sharpness of contrast between an optotype (52) and a black background (51); and measuring and calculating a spherical dioptric power and an astigmatism dioptric power of the eyes of the subject.

### REFERENCE TO RELATED APPLICATION

This application is a national stage application of International Patent Application No. PCT/CN2022/079250, filed on Mar. 4, 2022, which claims priority of Chinese Patent Application No. 202110083915.3, filed on Jan. 21, 2021, both of which are incorporated by reference herein in their entireties as part of this application.

### TECHNICAL FIELD

The present disclosure mainly relates to a field of optometry for refractive errors, and in particular, to a set of refractive test cards and measurement methods therefor.

### BACKGROUND

Refractive errors refer to that parallel light through the refractive action of an eye cannot form a sharp image on its retina because an image is formed in front of or behind the retina. Refractive errors include hyperopia, myopia, and astigmatism. All refractive error examinations require a complete set of instruments and equipment and professionally trained optometrists or ophthalmologists to conduct. Such examinations include:

1. Subjective Examination Method

A deviation caused by accommodation may be approximated but not completely counteracted. A refractive error examination after mydriasis may theoretically counteract the refractive error portion caused by accommodation. However, such corrected dioptric power of a subject with obvious accommodation obtained by post-mydriasis optometry does not represent subject's daily dioptric power, so the obtained optometry dioptric power often causes hazy visual acuity in daily life.

(a) Method for preliminarily analyzing and determining refractive properties according to visual acuity examination results (refractive error unquantifiable)

(b) Pinhole disk and slit film examination method (a simple method which tells us whether visual acuity may be improved by correcting the refractive errors, which is unquantifiable)

(c) Astigmatism chart optometry (only roughly estimating an astigmatic axis while the astigmatism dioptric power cannot be quantified)

(d) Cross cylindrical lenses and astigmatism corrector optometry (in which steps are complex and time-consuming, young subject is easily confused, and accuracy is affected by spherical dioptric power)

(e) Subjective refraction by inserting spherical lens only (in which the spherical dioptric power can be tested, but is affected by the presence of astigmatism dioptric power)

(f) Fogging method (which helps to better approach a true spherical dioptric power)

(g) Simultaneous sharpness method of red and green optotypes (which avoids exceeding the true spherical dioptric power)

(h) Laser speckle pattern method (which is not common, and requires a high device cost)

2. Objective Examination Method

(a) Direct ophthalmoscopy (in which only the dioptric power of glasses worn at that time may be known, but the latest dioptric power cannot be known, and whether the dioptric power of the glasses fitting accurately cannot be known)

(b) Keratometer (by which only a corneal curvature may be obtained to reckon the spherical dioptric power formed by a cornea and astigmatism and an astigmatic axis caused by asymmetry. However, the spherical dioptric power, the astigmatism dioptric power, and the astigmatic axis of the whole eye cannot be known)

(c) Autorefractor (through which the spherical dioptric power, the astigmatism dioptric power, and the astigmatic axis may be roughly estimated, however, due to the influence of involuntary accommodation of the subject, the accuracy of detected dioptric power is often inconsistent with a true dioptric power)

(d) Strip light retinoscopy (which is generally matched with lenses in an examination, so as to determine an approximate dioptric power when neutralization is reached, and the spherical dioptric power, astigmatism dioptric power, and astigmatic axis obtained through which are estimated values, and therefore the lens insertion photometry and a cross cylindrical lenses and astigmatism corrector optometry are also needed to find the spherical dioptric power, the astigmatism dioptric power, and the astigmatic axis.)

(e) Retinoscopy

In addition, in ophthalmology, astigmatism is a manifestation of refractive error of eyes. Most astigmatism is related to the curvature of the cornea. When parallel light enters the astigmatic eye which has unequal refractive power on different meridians, the light on each meridian cannot be focused at one point (a focal point). The same optotype will therefor form more than one object images which do not overlap completely, thus not forming a clear object image. This condition is referred to as astigmatism.

Most clinical astigmatism is simply caused by asymmetric curvature of the cornea, but sometimes, the clinical astigmatism is caused by eye lesions, particularly, anterior segment lesions, for example, ptosis of an upper eyelid, ocular conjunctival mass compression, corneal scab, pterygium, the crystalline lens' shape, position, and opacity and the like.

Generally, the existing astigmatism examination usually adopts the following methods:

1. Visual Acuity Examination

Astigmatism may be found through distant vision and near vision examinations. A patient with severe astigmatism has poor distant vision and near vision.

2. Astigmatism Chart Observation

Subjective observation of astigmatism chart can be used, based on the relatively sharp or hazy image on a retina to detect preliminarily the presence of an astigmatism meridian.

The principle is based on a visual effect of multi-point concatenation:

A commonly used fixed astigmatism chart (a clock type astigmatism chart) consists of a plurality of radial lines, and each line is based on the principle of multi-point concatenation. The more the points, the sharper the concatenation. When there is no astigmatism, of course, each radial line of the chart has its own axis and is different from axis of other lines. However, the concatenation densities of the points along the axis of all individual lines are the same, so the sharpness of the lines looks the same, and there is no individual prominent line. When there is astigmatism, since each line has its own axis which is different from each other, the concatenation density of the points along the axis of individual lines is different. At this moment, one or two lines appear to have relatively high and obvious sharpness. The concatenation along the axis of the line is relatively dense concatenation of more points, so the line looks sharper. The concatenation at 90° relative to the axis of the line is relatively loose concatenation of less points, so the line looks hazy.

FIG. 1 and FIG. 2 are examples, which show a traditional clock type astigmatism chart under observation.

FIG. 1 shows that, when there is no astigmatism, the radial linear optotypes arranged divergently appear to be equally sharp along individual axis.

FIG. 2 shows that, when there is astigmatism, for example, with an astigmatic axis of 90°. When the deviation from the astigmatic axis of 90° decreases, the corresponding radial linear optotypes are getting sharper, and is sharpest at zero deviation. In contrast, as the deviation from the astigmatic axis increases, the corresponding radial linear optotypes are getting more diffused and hazy.

Why a vertical radial linear optotype is the sharpest? Because all points in the line converge along the axis of the vertical radial linear optotype, an observer thus sees a very sharp vertical line when many focusing lines along the vertical axis overlap. Once the observer is corrected by a corresponding astigmatic lens, the sharpness of each line in FIG. 2 seen by the observer is the same as that in FIG. 1.

3. Retinoscopy:

Specifically, it is prompted that the subject has astigmatism in case of one of the following five conditions: (1) different widths of reflective light bands, (2) different dioptric powers of a pair of meridians, (3) irregular fundus light reflection, (4) scissors movement, and (5) inconsistent movement directions of retinoscope strip light and a fundus reflective light band.

4. Dioptric Power Examination

(a) Objective Optometry

Measurement methods for the astigmatism include a cylindrical lens method and a spherical lens method. An astigmatic axis and astigmatic dioptric power can be measured. The astigmatism may be classified as mild astigmatism (≤2.00 DC), moderate astigmatism (2.25˜4.00 DC), and severe astigmatism (≥4.00 DC) according to the astigmatic dioptric power. The astigmatism which is lower than 1.00 DC belongs to physiological astigmatism.

(b) Subjective Refraction Using Trial Lenses

The subjective refraction using trial lenses is generally performed after objective optometry.

5. Corneal Astigmatism Examination

The corneal astigmatism examination involves a keratometer, corneal topography, or a subjective astigmatism testing using cylindrical lenses.

6. Fundus Examination

An optic disc papilla is usually oval. For a person with high astigmatism, the vertical edge of the optic papilla may appear sharper, while the horizontal edge looks blurry or vice versa. From such relative sharpness and blurriness of the optic disc papilla edges, the astigmatic axis can be inferred approximately. The accuracy of the above refractive error examination methods are affected by many factors. The following are the disadvantages and shortcomings:

First, the subject has to go to places such as a hospital, a clinic, and an optical store to have a face-to-face optometry examination by a professional eye doctor or optometrist.

Second, examination sites need to have a large number and varieties of optometry and optical lens instruments and equipments.

Third, the examiner needs to have sufficient professional optical optometry principle knowledge and proficient practical operation skills.

Fourth, the examination time is affected and limited by the daily life and office hours available to the examiner and the subject, as the traffic on the way back and forth is time-consuming and laborious.

Fifth, in conventional automatic computer refractometer, due to the close proximity between the eyes and the optotypes, proximal accommodation effect is induced automatically, resulting in an inaccurate computer optometry result.

Sixth, conventional subjective refraction by inserting spherical lens only requires an eye and a lens to operate together at a fixed close distance. This prerequisite is often not strictly obeyed by examiners and subjects. Meanwhile, the most appropriate spherical dioptric power is not known during insertion lens optometry, so the trial lens often needs to be replaced during optometry, which easily induces involuntary accommodation of the eyes of the subject, hence resulting in inaccurate dioptric power measurements. In addition, the examination method is a complex process, and the subjects, especially young children, do not understand what to do to cooperate with the examiner for optometry, so an inaccurate optometry result with significant deviation is often obtained. Quite often, in a busy pediatric ophthalmology department, the examiner may skip this conventional insertion lens optometry to directly use the dioptric power obtained from automatic computer refractometer, thus making a bigger error.

Seventh, the existing astigmatism chart examination process is complex, and is conducted under the guidance of a professional with an objective examination in a special place, rendering the astigmatism chart clumsy to use. In addition, this method is conducted only after partial correction of the subject' refractive error that the radial strips appear relatively sharp. Ibis method cannot be conducted without the refractive error being partly corrected with lens first.

With respect to the above optometry methods and influencing factors, the optometry results of the same subject often deviate at different places. Deviation of results is also observed in the same subject subjected to optometry by the same optometrist or ophthalmologist at the same place on different dates that are close to one another.

Currently, commonly used visual acuity charts in clinical practice include: an International Standard Visual acuity chart, a Landolt C visual acuity chart, a Logarithmic visual acuity chart, a Digital visual acuity chart, a Snellen visual acuity chart, and a children's Graphical visual acuity chart.

Commonly used astigmatism charts include: a fixed astigmatism chart (a clock astigmatism chart), and a movable astigmatism chart (a fan block optotype consisting of a fan-shaped radial marking line and a rotatable disk, on which there are a group of grid-shaped blocks which are perpendicular to each other, and an inverted V-shaped optotype).

Regardless of the types of visual acuity charts and astigmatism charts, there is a problem of too many optotypes. Presence of many optotypes causes crowding effect when the subject is observing and affects the judgment of the subject. At this moment, the subject is prone to increase accommodation involuntarily, resulting in a higher spherical dioptric power lens is required for obtaining a sharp image, meaning that the spherical dioptric power is easily overestimated.

In an environment of a formal optometry site, it is not easy for the subject to relax, and an unnecessary accommodation is easily induced, resulting in erroneous optometry results.

The insertion lens optometry method needs to replace or rotate insertion lenses frequently, which easily distract and interfere the subject, causing accommodation changes and associated error in the measurement of the dioptric power.

### SUMMARY

When a spherical dioptric power, an astigmatism power, and an astigmatic axis are measured using the above test tools and methods, there is inconvenience in use as well as subjective and empirical deviation during refraction among different optometrists or eye doctors, and thus a new set of refractive test tools and measurement methods are developed.

A technical problem to be solved by the present disclosure is to provide an innovative simple optometry tool and method for determining whether a subject has a refractive error, which type of refractive error, and quantification of the refractive error.

In order to satisfy the above technical demands, the present disclosure provides a refractive test card, including an optotype under a black background. The optotype includes a partition unit at a central position. The partition unit visually separates the optotype.

In an embodiment of the present disclosure, the optotype includes a strip-shaped optotype. Color of the strip-shaped optotype includes either of white and red.

In an embodiment of the present disclosure, the optotype includes a cross-shaped optotype. Color of the cross-shaped optotype includes white.

In an embodiment of the present disclosure, a shape of the partition unit includes a rectangle or a circle.

In an embodiment of the present disclosure, the optotype has a length of 260 mm±50 mm, and a width of 5 mm 2 mm.

In an embodiment of the present disclosure, the partition unit has a width of less than or equal to 5 mm, and a height range of 5 mm 2 mm.

In an embodiment of the present disclosure, the refractive test card includes an astigmatism test card and a dioptric power test card. The astigmatism test card includes a white strip-shaped optotype and a cross-shaped optotype. The dioptric power test card includes a red strip-shaped optotype.

The present disclosure further provides a test method for implementing the refractive test card, including:
step a, determining whether a subject has astigmatism based on the sharpness of contrast between an optotype and a black background of an astigmatism test card observed by the subject;
step b, in response to determining that the subject has astigmatism, determining an astigmatic axis; and in response to determining that the subject does not have astigmatism, turning directly to step d;
step c, determining whether the subject has myopia or hyperopia based on sharpness of contrast between an optotype and a black background of a spherical dioptric power test card observed by the subject;
step d, testing, measuring and calculating a spherical dioptric power of the subject; and
step e, testing, measuring and calculating an astigmatism dioptric power of the subject in a case that the subject has astigmatism.

In an embodiment of the present disclosure, determining the astigmatic axis in step b includes:
step b1, testing an unilateral naked eye of the subject by rotating the astigmatism test card at a speed no higher than 12.5°/second, to obtain the maximum sharpness axial angle of the astigmatism test card observed by the subject; and
step b2, determining the maximum sharpness axial angle ±90° as the astigmatic axis.

In an embodiment of the present disclosure, step d further includes:
step d1, by moving the unilateral naked eye of the subject towards the spherical dioptric power test card, thus obtaining the distance d on seeing the sharpest optotype image of the spherical dioptric power test card meaning that the optotype is in focus. Therefore, this distance d is equal to focal length f:

f=d (1);

step d2, obtaining a first spherical dioptric power D₁ according to the relation between f and spherical dioptric power D:

D=1/f (2);
step d3, rotating the spherical dioptric power test card by 90°, and repeating steps d1 and d2 to obtain a second spherical dioptric power D₂;

step d4, calculating the spherical dioptric power D of one eye of the subject:

D=(D₁+D₂)/2 (3); and
step d5, repeating steps d1˜d4 to obtain the spherical dioptric power of the other eye.

In an embodiment of the present disclosure, step e further includes:
step e1, placing the spherical dioptric power test card in the maximum sharpest axial angle of step b1;
step e2, obtaining the sharpest image distance d and the focal length f by moving the unilateral naked eye of the subject towards the spherical dioptric power test card:

f=d (1);
step e3, obtaining the first spherical dioptric power D₁ according to the relation between f and spherical dioptric power D:

D=1/f (2);
step e4, placing the spherical dioptric power test card in the astigmatic axis, and repeating steps e1˜e3 to obtain a second spherical dioptric power D₂;
step e5, calculating the astigmatism dioptric power D′ of the eye of the subject:

D′=D₂-D₁ (3); and
step e6, repeating steps e1˜e5 to obtain the astigmatism dioptric power of the other eye.

In an embodiment of the present disclosure, in step a:

rotating the astigmatism test card; and in a case that the subject observes that there is no change in the sharpness of contrast between the optotype and the black background, thus determining that eye of the subject does not have astigmatism; and otherwise, determining that the subject has astigmatism.

Compared with the conventional art, the present disclosure does not need to use a concave spherical lens for correcting myopia during refraction. When the subject sees the optotype clearly, a focusing line in myopic eye is moved backward to just overlap the retina, which represents that a distance between the eye and the optotype is equal to the focal length. Therefore, the spherical dioptric power and the astigmatism dioptric power are obtained, a traditional optometry refraction method is greatly simplified, and the type and the quantification about refractive errors are detected and determined very conveniently and quickly.

According to the test methods of the above claims, a close concave lens is not used, thereby eliminating unnecessary refractive error caused by accommodation surge induced by close operation distance between lens and eye in conventional optometry.

### BRIEF DESCRIPTION OF THE DRAWINGS

The drawings are included to provide a further understanding of this application. They are included and form a part of this application. The drawings show the embodiments of this application and play a role in explaining the principles of the present disclosure together with the description. In the drawings:

FIG. 1 is a schematic diagram of a linear optotype seen by a subject without astigmatism with a traditional clock type astigmatism chart;

FIG. 2 is a schematic diagram of a linear optotype seen by the subject with astigmatism with the traditional clock type astigmatism chart;

FIG. 3 is a schematic diagram of compositions of an astigmatism test card 30 in a first embodiment of the present disclosure;

FIG. 4 is a schematic diagram of compositions of an astigmatism test card 40 in a second embodiment of the present disclosure;

FIG. 5 is a schematic diagram of compositions of a spherical dioptric power test card 50 in a third embodiment of the present disclosure;

FIG. 6A and FIG. 6B are presentation comparison diagrams of a cross-shaped optotype at different angular positions during testing;

FIG. 7A and FIG. 7B are presentation comparison diagrams of a cross-shaped optotype at different angular positions during testing;

FIG. 8A and FIG. 8B are presentation comparison diagrams of a cross-shaped optotype at different angular positions during testing;

FIG. 9 is a schematic diagram of light focusing of the subject with only astigmatism;

FIGS. 10A-10C are views on retinas during an astigmatism test;

FIG. 11 is a flowchart of a complete test method for implementing a test card of the present disclosure;

FIG. 12 is a schematic diagram of light focusing when the subject with a spherical dioptric power refractive error looking at an optotype during testing;

FIG. 13 is a detailed flowchart of step 1 in FIG. 11;

FIG. 14 is a detailed flowchart of step 3 in FIG. 11; and

FIG. 15 is a detailed flowchart of step 4 in FIG. 11.

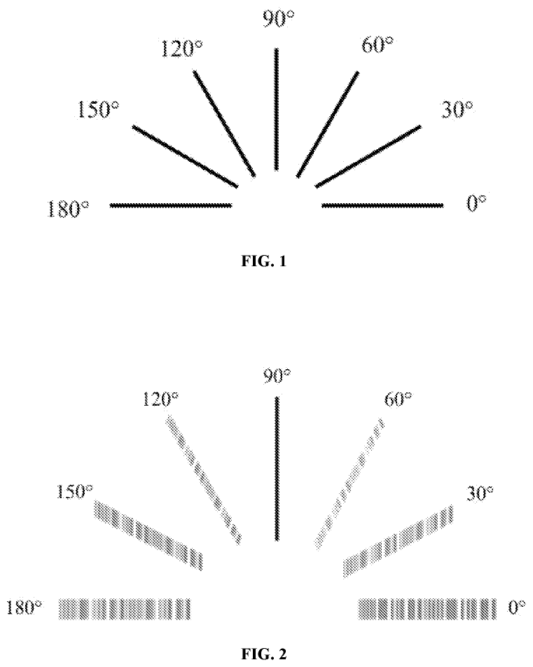

### REFERENCE NUMERALS IN THE DRAWINGS

04—eye
30—astigmatism test card
31—black background
32—white strip-shaped optotype
33—partition unit
40—astigmatism test card
41—black background
42—white cross-shaped optotype
43—partition unit
50—spherical dioptric power test card
51—black background
52—red strip-shaped optotype
53—partition unit

### DETAILED DESCRIPTION OF THE EMBODIMENTS

To describe technical solutions in embodiments of this application more clearly, the following briefly describes the drawings required for describing the embodiments. Apparently, the drawings in the following description are merely some examples or embodiments of this application. This application is also applied by the ordinary skill in the art to other similar situations based on these drawings without any creative efforts. Unless apparent from the language environment or otherwise stated, like reference numerals in the drawings refer to like elements or operations.

As shown in this application and the claims, terms such as “a”, an”, and/or “the” do not refer in particular to a singular form but may also include a plural form, unless exceptional cases are clearly indicated in the context. In general, terms “include” and “contain” only indicate inclusion of steps and elements that are clearly identified, and these steps and elements do not form an exclusive enumeration, and a method or device may also include other steps or elements.

Unless otherwise specified, relative arrangements of components and steps elaborated in these embodiments, numeric expressions and numeric values do not limit the scope of this application. Furthermore, it is to be understood that, for the convenience of descriptions, the size of each part shown in the drawings is not drawn in accordance with an actual proportional relation. Technologies, methods, and devices known by those skilled in the related art may not be discussed in detail. However, where appropriate, the technologies, the methods, and the devices shall be regarded as part of the description. In all examples shown and discussed herein, any specific values shall be interpreted as only exemplar values instead of limited values. As a result, other examples of the exemplar embodiments may have different values. It is to be noted that similar marks and letters represent similar items in the following drawings. As a result, once a certain item is defined in one drawing, it is unnecessary to further discuss the certain item in the subsequent drawings.

In the descriptions of this application, it should be understood that orientation or position relations indicated by “front, back, up, down, left, and right”, “lateral, longitudinal, vertical, and horizontal”, “top and bottom” and the like are generally based on the orientation or position relations shown in the drawings, which are intended to describe this application and to simplify the descriptions. Where not otherwise specified, they do not indicate or imply that the referring device or element must have a specific location and must be constructed and operated with the specific location, and accordingly they cannot be understood as limitations to this application. The orientation terms “inner and outer” refer to the inner and outer relative to the contours of each component itself.

For the purpose of description, spatial relative terms such as “over”, “above”, “on an upper surface” and “upper” may be used herein for describing a spatial position relation between a device or feature and other devices or features shown in the drawings. It should be understood that the spatial relative terms are intended to contain different orientations in usage or operation besides the orientations of the devices described in the drawings. For example, if the devices in the drawings are inverted, devices described as “above other devices or structures” or “over other devices or structures” will be located as “below other devices or structures” or “under other devices or structures”. Thus, an exemplar term “above” may include two orientations namely “above” and “below”. The device may be located in other different modes (rotated by 90 degrees or located in other orientations), and spatial relative descriptions used herein are correspondingly explained.

In addition, it is to be noted that terms “first”, “second” and the like are used to limit parts, and are only intended to distinguish corresponding parts. If there are no otherwise statements, the above terms do not have special meanings, such that they cannot be understood as limits to the scope of protection of this application. In addition, although the terms used in this application are selected from the well-known and common terms, some of the terms mentioned in the description of this application may be selected by the applicant according to his or her discretion, and their detailed meanings are described in the relevant parts of the description herein. In addition, it is required to understand this application not only through the actual terms used, but also through the meaning of each term.

In this application, the flowchart is used to describe operations executed by a system according to the embodiments of this application. It is to be understood that previous or subsequent operations are not always executed accurately in sequence.

Instead, various steps may be processed in an inverted sequence or simultaneously. In addition, other operations may also be added to these processes, or one or more operations may be removed from these processes.

### Embodiment 1

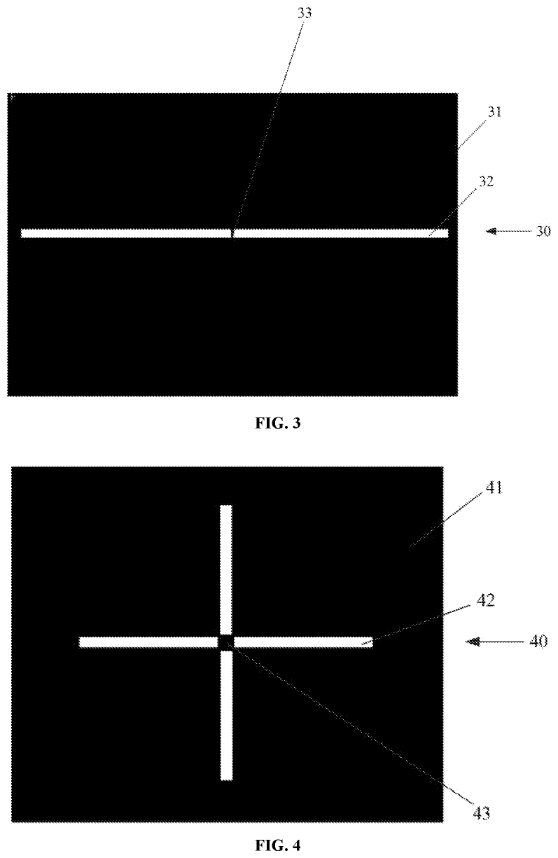

FIG. 3 is a schematic diagram of an astimatism test card of a first embodiment of the present disclosure.

The test card 30 is an astimatism test card consisting of a black background 31 and a single white strip-shaped optotype 32, and is not limited to a combination of white and strip shape mentioned above, but requires that:
first, the test card 30 has the black background 31; and
second, a horizontal white strip-shaped optotype 32, with a length of 260 mm and a width of 5 mm, is provided in the middle of the test card 30.

A partition unit 33, with a width of 1.5 mm and a height of 5 mm, is arranged in the center of the white strip-shaped optotype 32. The partition unit 33 is not limited to a rectangle, a circle, or other shapes.

Under the black background 31, the contrast of black and white is very strong and obvious at an edge. This is beneficial for the subject to easily distinguish between ambiguity and clarity at a relatively long distance. This is very suitable for judging an astigmatism axis direction.

Therefore, the astigmatism strip test card 30 with the above structure is an embodiment.

### Embodiment 2

FIG. 4 is a schematic diagram of an astigmatism test card of a second embodiment of the present disclosure.

The test card 40 is an astigmatism test card consisting of a black background 41 and a single white cross-shaped optotype 42, and is not limited to a combination of white and strip shape mentioned above. A deformation on this basis requires:
first, the background of the test card is black;
second, two strips which are at an angle of 90° are provided in the middle of the test card;
third, each strip has the length of 260 mm, and the width of 5 mm; and
fourth, in order to avoid a density effect, a partition unit 43 is specially arranged in a center of a cross-shaped optotype 42. The partition unit 43 is a square with a side length of 6 mm.

The reason for selecting the white astigmatism strip in the foregoing two embodiments is that:

Under the black background, the contrast of black and white is very strong and obvious at the edge. This is beneficial for the subject to easily distinguish between ambiguity and sharpness at the relatively long distance. This is very suitable for judging the astigmatic axis.

### Embodiment 3

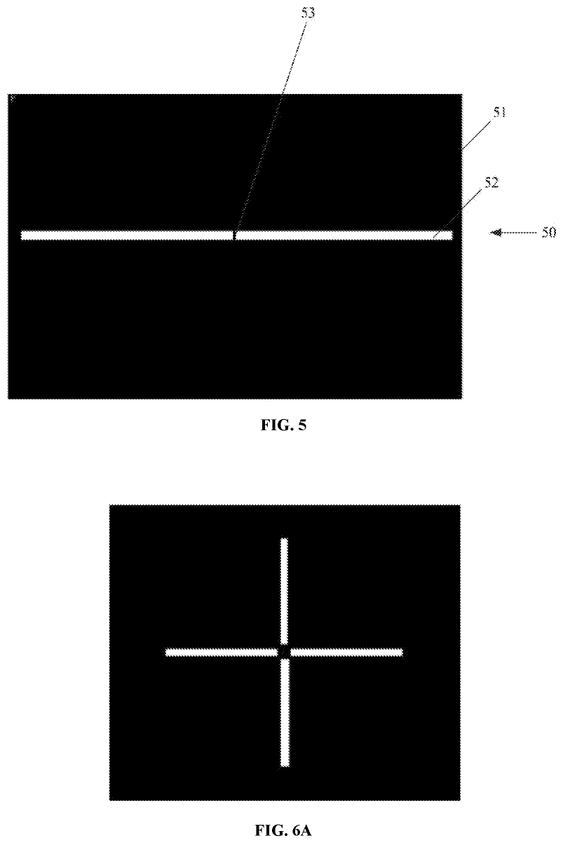

FIG. 5 is a schematic diagram of an optotype card of a third embodiment of the present disclosure.

The test card 50 is an optotype spherical dioptric power test card consisting of a black background 51 and a single red strip-shaped optotype 52, and is not limited to a combination of red and strip shape mentioned above, but requires that:
first, the background of the test card 50 is black;
second, a horizontal red strip-shaped optotype 52, with a length of 260 mm and a width of 5 mm, is provided in the middle of the test card 50; and
third, a black rectangular partition unit 53, with a width of 1.5 mm and a height of 5 mm, is arranged in the center of the red strip-shaped optotype 52. The partition unit 53 is not limited to a rectangle, a circle, or other shapes.

The reason for selecting the red spherical dioptric power optotype is that:

In clinical optometry methods, a measurement deviation of the spherical dioptric power between red light and green light is about 0.5 DS. The red optotype can counteract an involuntarily increased spherical dioptric power caused by proximity induced accommodation and convergence because the formed visual image is relatively backwardly positioned and is closer to the retina. In contrast, the green optotype cannot counteract the involuntarily increased spherical dioptric power caused by proximity induced accommodation and convergence because the green optotype image is relatively forwardly positioned and is further away from the retina. Based on this reason, a red spherical dioptric power optotype is applied to a unique optometry method of the present disclosure.

A basic process for testing a refractive error by using the test card in the above embodiment is as follows:

The astigmatism test card 30 or 40 is applied to test whether there is an astigmatic refractive error that needs to be corrected clinically. If there is an astigmatic refractive error, it needs to be determined first.

FIG. 11 illustrates main process steps of the test method of the present disclosure, including steps 1-4.

In step 1, the astigmatism test card 30 or 40 is used for testing whether a subject has astigmatism.

In step 2, if it is determined that the subject has clinically significant astigmatism, an astigmatic axis is determined.

In step 3, a myopia/hyperopia dioptric power is determined and obtained by using the dioptric power test card 50.

In step 4, for a subject with the astigmatism, the astigmatism dioptric power is obtained by the difference between a strongest dioptric power and a weakest dioptric power.

In the above test, final results of the subject include the following four cases:
Case 1: the subject has neither astigmatism nor myopia or hyperopia;
Case 2: the subject has astigmatism, but does not have clinically significant myopia or hyperopia, and relevant parameters of the astigmatism are obtained through step 2 and step 3;
Case 3: the subject does not have astigmatism, but has myopia or hyperopia, and relevant parameters of the myopia or hyperopia are obtained through step 3; and
Case 4: the subject has both astigmatism and myopia or hyperopia, and relevant parameters including the astigmatism and the myopia or hyperopia are obtained through steps 2, 3, and 4.

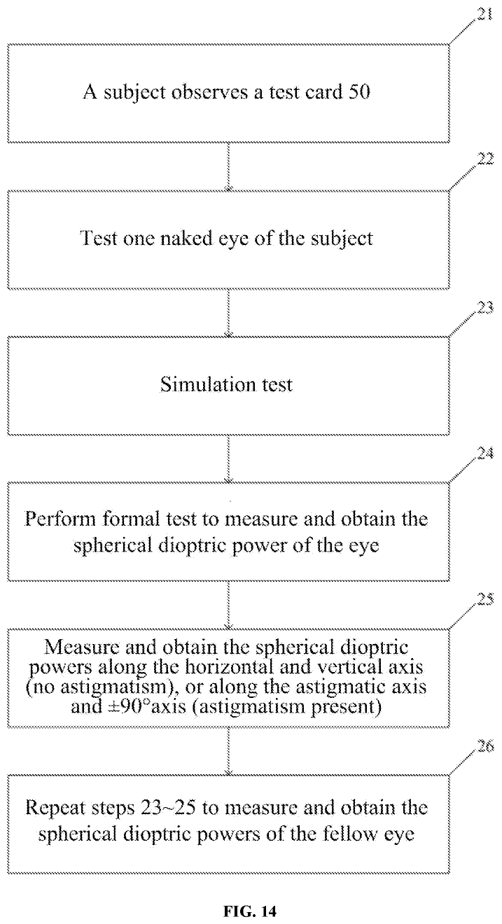

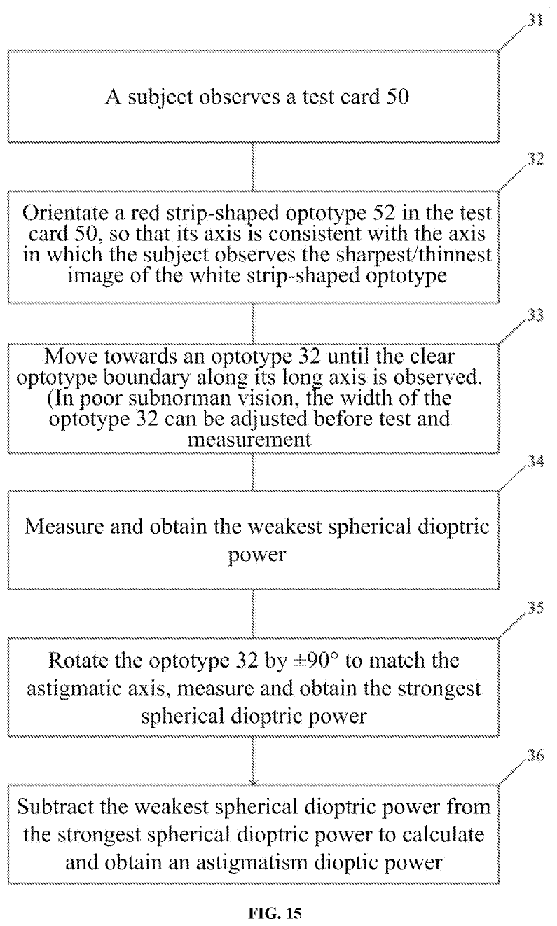

The test method for using the refractive test card of the present disclosure and detailed processes of the above steps are introduced below in combination with FIG. 11, FIG. 13, FIG. 14, and FIG. 15.

A method for determining whether the subject has the clinically significant astigmatism in step 1 includes step 11, step 12, step 13 and step 14.

In step 11, at a distance of about 5 meters from the astigmatism test card 30, the subject looks at the astigmatism test card 30 with the single white strip-shaped optotype 32 under the black background 31.

The test card 30 adopts artificial illumination, for example, a direct illumination method; and the illumination is not lower than 300 lx, and the illumination is required to be uniform, constant, non-reflective, and dazzle-free.

In step 12, a unilateral naked eye of the subject is tested; and generally, a right eye is tested first, and then a left eye is tested.

When one eye is tested, the other eye needs to be covered. The head of the subject needs to be kept upright and cannot skew. During testing, the subject may naturally blink to ensure that eyes are moist and not dry to avoid affecting the sharpness of the visual acuity due to dryness.

In step 13, the test card 30 is rotated counterclockwise slowly.

In step 14, whether the subject has astigmatism is determined and an astigmatism axis direction is determined according to the viewing response of the subject.

When the subject does not have the clinically significant astigmatism, it represents that the subject has only a single spherical dioptric power, so there is only one focusing line. At this moment, when the test card 30 is rotated to any direction, the clarity and ambiguity of contrast of the white strip-shaped optotype 32 and the black background 31 seen by the subject are similar. There is no case where a certain direction is particularly clear, and it indicates that the subject does not have clinically significant astigmatism.

In an opposite case, in a process of rotating the test card 30, the clarity of contrast of the white strip-shaped optotype 32 and the black background 31 seen by the subject changes, and it can be determined that the subject has clinically significant astigmatism. If the subject cannot see clearly, the subject should move slowly to get close to the white strip-shaped optotype with small steps, and to stop to start testing at a place where an obvious strip-shaped optotype can be seen.

Some subjects may be insensitive to rotation, and it is not easy for them to distinguish the visual changes of the white strip-shaped optotype 32 during rotation.

For such subjects, the test card 40 of the white cross-shaped optotype 42 in Embodiment 2 will replace optotype 32.

The method steps are the same as above. In step 14, the determination is performed based on whether, starting from a horizontal line and a vertical line, the two strips look similar in clarity, or one is hazier than the other.

Assuming that the strip in a 0°˜180° direction is hazy, and the strip in 90°˜270° direction is clear, an accurate position of the astigmatism axis may be found by slowly rotating within a range of ±10° counterclockwisely and clockwisely around the 90°˜270° axial direction.

If the horizontal line and the vertical line look similar without significant differences, it represents that the astigmatism axis is not on these two lines. The white cross-shaped optotype 42 is placed at four strips respectively corresponding to 45° and 135°, 225° and 315° respectively. It will be known again whether the subject seeing the two strips in each set with similar clarity, or one strip is hazier than the other strip again.

If the subject still thinks that there is no difference between the two strips in each set, the white cross-shaped optotype 42 is placed at four strips respectively corresponding to 22.5° and 112.5°, 202.5° and 292.5°. The subject is asked for the same questions again. By analogy, the white cross-shaped optotype 42 may also correspond to 11.25° and 101.25°, 191.25° and 281.25°, and the like respectively.

The rule is that: the two strips of the white cross-shaped optotype 42 are perpendicular to each other all the time, while the angular position from the horizontal axis varies and is adjusted sequentially during each display.
90°/2 getting 45°
90°/4 getting 22.5°
90°/8 getting 11.25°
90°/16 getting 5.625°
90°/32 getting 2.8125°.

At certain angle of deviation from the horizontal axis, where the subject observed that the white cross-shaped optotype 42 has one hazy strip and one clear strip. After this angular position from the horizontal axis is found, the exact astigmatism axis is found via step 13, this time slowly rotating within a range of 10°, alternating counterclockwisely and then clockwisely.

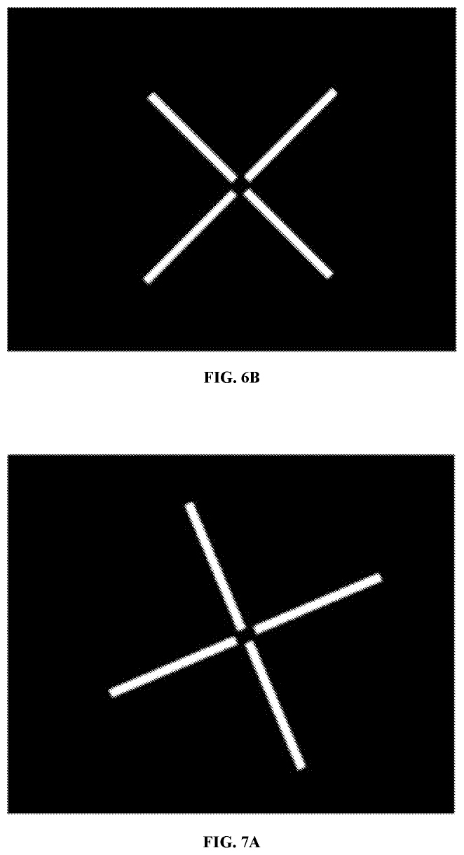

FIG. 6A and FIG. 6B-FIG. 8A and FIG. 8B respectively show comparison diagrams of the cross-shaped optotype 42 at the above different angular positions during testing.

That is, the cross-shaped optotype as shown in FIG. 6A corresponds to 0° and 180° axis, 90° and 270° axis respectively.

The cross-shaped optotype as shown in FIG. 6B corresponds to 45° and 225° axis, 135° and 315° axis respectively.

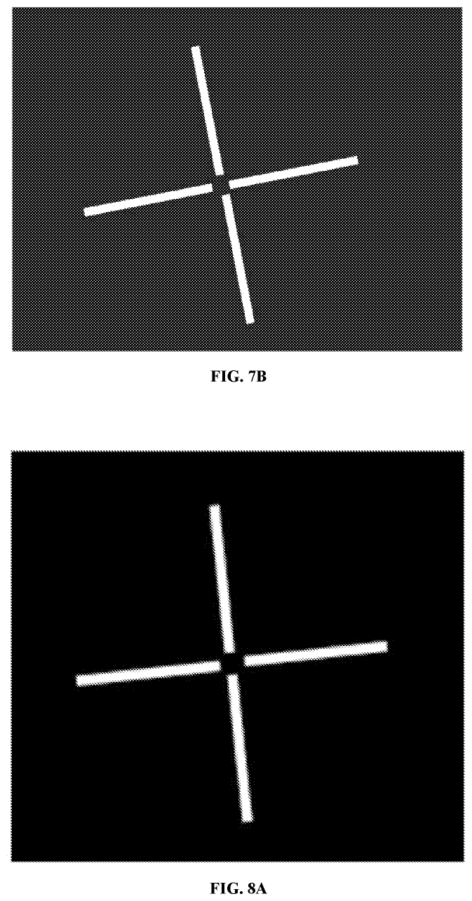

The cross-shaped optotype as shown in FIG. 7A corresponds to 22.5° and 202.5° axis, 112.5° and 292.5° axis respectively.

The cross-shaped optotype as shown in FIG. 7B corresponds to 11.25° and 191.25° axis, 101.25° and 281.25° axis respectively.

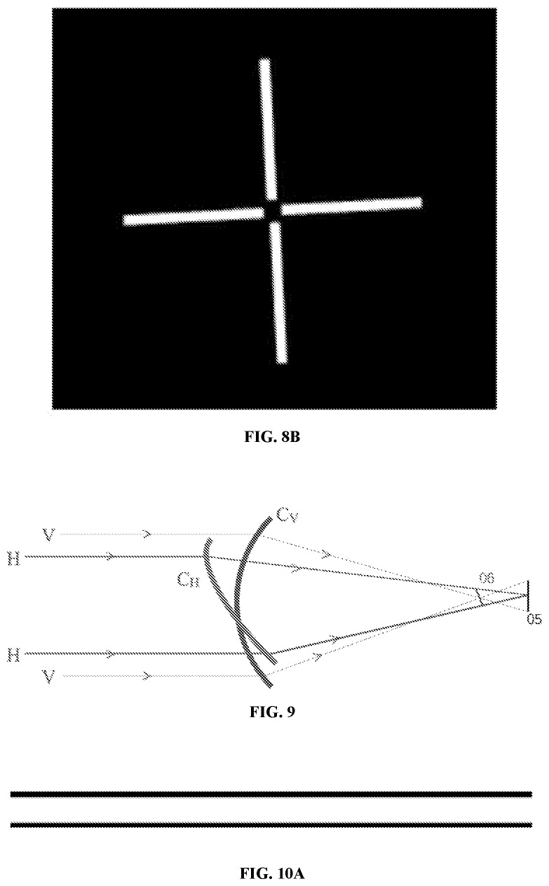

The cross-shaped optotype as shown in FIG. 8A corresponds to 5.625° and 185.625° axis, 95.625° and 275.625° axis respectively.

The cross-shaped optotype as shown in FIG. 8B corresponds to 2.8125° and 182.8125° axis, 92.8125° and 272.8125° axis respectively.

If the subject determines that the astigmatism test card 30 does not change in clarity at any position, it indicates that there is no clinically significant astigmatism. If the astigmatism test card 40 does not have any change in clarity at any angular position no matter the subject is at any position, that is, in any case, the two strips of the cross-shaped optotype have the same clarity or ambiguity without difference, then it is determined that the subject does not have clinically significant astigmatism.

Returning to step 1, when it is determined that the subject does not have clinically significant astigmatism in step 1, and then the subject may have a simple spherical refractive error only. At this moment, step 2 is skipped, in step 3 dioptometry of myopia or hyperopia is directly applied where the subject will look at the optotype spherical dioptric power test card 50 with the single red strip-shaped optotype 52 under the black background 51.

It is to be noted that, for a subject with myopia but without clinically significant astigmatism has only one focusing line and the focusing line is in front of the retina, the image will be hazy. Traditionally, at this moment, a general optometry method is used for finding a suitable concave spherical lens for correcting myopia to move this focusing line back onto the retina to obtain a clear image, that is, the focusing line overlaps the retina. At this moment, a good visual acuity effect may be achieved.

The present disclosure has the following originalities:

The concave spherical lens for correcting myopia does not need to be used, but the subject moves forward to the optotype until the subject just sees the clear optotype. At this moment, the focusing line moves backward to just overlap the retina, which also represents that the distance between an eye and the optotype is equal to the focal length. This distance may be used for finding the spherical dioptric power of the subject's refractive error.

Further, specific method steps for testing the spherical dioptric power of refractive error are described in detail below in combination with FIG. 14, which includes step 21, step 22, step 23, step 24, step 25 and step 26.

In step 21, at a distance of about 5 meters from the subject, the spherical dioptric power test card 50 with the single red strip-shaped optotype 52 under the black background 51 is shown to the subject.

The test card 50 adopts artificial illumination, for example, a direct illumination method; and the illumination is not lower than 300 lx, and the illumination is required to be uniform, constant, non-reflective, and dazzle-free.

Avoid direct sunlight or strong light illumination to the test card

In step 22, a unilateral naked eye of subject is tested; and generally, a right eye is tested first, and then a left eye is tested.

When one eye is tested, the other eye needs to be covered. The head of the subject needs to be kept upright and cannot skew. The subject may naturally blink to ensure that eyes are moist and not dry to avoid affecting the clarity of the visual acuity due to dryness.

In step 23, a simulation test is performed first, and the purpose thereof is to avoid the subject from using unwanted additional accommodation before reaching or exceeding the focusing distance, which affects a test result. During the simulation test, an eye needs to be covered, and the subject moves forward slowly to get close to the single red strip-shaped optotype 52 under the black background with small steps from the distance of about 5 meters, such that the subject sees that the optotype boundary along its long axis is hazy first, then becoming relatively clear, and finally completely clear. At this moment, the subject continues to move forward and then slowly moves backward to observe, distinguish, and perceive the change in the clarity of the optotype.

In step 24, when a formal test starts, the subject moves forward slowly to get close to the single red strip-shaped horizontal optotype 52 under the black background 52 with small steps from the distance of about 5 meters with an eye covered, and stops moving immediately once the clear optotype boundary along its long axis is observed. It is to be noted that the subject once moving forward cannot move backward during the process. If the subject cannot distinguish a position where the optotype image changes from hazy to clear due to too big and too fast steps, the subject needs to moves back to a starting point to restart with smaller steps and slower speed.

FIG. 12 is a position of this testing process, that is, a schematic diagram of light focusing when an eye without astigmatism sees the optotype.

In FIG. 12, 04 represents the eye, C represents the cornea, M is the retina, O represents the optotype, and f represents the focal length which equals the distance d between the subject and the optotype when the subject just observed a clear optotype image.

When the subject sees the optotype O at a distance of greater than or equal to 6 meters, the light from each same point of the optotype O is parallel on reaching the cornea C, and the spherical dioptric power in the horizontal direction is 0 DS, so the light gathering power is the weakest and the light in the horizontal direction is focused to a rear focusing line. At this moment, the rear focusing line just overlaps the retina M, when the subject sees the optotype O clearly, the numerical value of the distance d is recorded, and d is the distance from the eye 04 of the subject to the optotype O. At this moment:

d=f (1)

Where, f is the focal length in meters.

In order to improve the accuracy, the subject needs to be tested repeatedly for five times in the same refraction assessment. Since it is possible that a subjective accommodation status of the subject changes during refraction process, the refraction measurement needs to be performed several times to obtain a mean value to improve the accuracy by reducing the error induced by the fluctuation of the accommodation status. The mean value of f is obtained from the numerical values of five d measurements.

After the assessment and measurement in the horizontal direction is completed, the assessment and measurement in the vertical direction is started, and the test card is rotated by 90°, so that the single red strip-shaped optotype 52 under the black background is vertical, and steps 23 and 24 are repeated.

According to the formula for the spherical dioptric power:

D=1/f (2)

Where, D is a spherical dioptric power, and is also referred to as a dioptric power.

When the subject sees the optotype O clearly, f is just equal to d, so the spherical dioptric power of the subject may be calculated according to the numerical value of d.

In step 25, a mean value of spherical dioptric power is obtained according to d measurements obtained in horizontal axis and vertical axis or an astigmatism axis and ±90° axis respectively.

The mean value of D obtained by the single red strip-shaped optotype 52 under the black background in the horizontal axis and the vertical axis is taken as the dioptric power D of this eye, or the mean value of the D obtained by the astigmatism axis and ±90° axis is taken as the dioptric power of this eye. (The difference between the horizontal axis and the vertical axis is not greater than 0.25, if the difference is greater than 0.25, then the subject has astigmatism and step 2 is repeated to obtain the astigmatism axis, and the corresponding d and D are measured and obtained by step 21 along the astigmatism axis and ±90°.)

In step 26, after measurement for dioptric power in one eye is completed, the above steps 23˜25 are repeated to the other eye to obtain its dioptric power.

For steps 21˜26, in combination with FIG. 12, a calculation process of the dioptric power is described by examples:

The following table data records the test values of the distance d, and the calculation results of the dioptric power.

In a case of myopia, “−” needs to be added in front of the dioptric power.

Referring the example of the following table 1:

| | d (m) | f (m) | D = 1/f | Mean value of D |
|---|---|---|---|---|
| Horizontal | 0.460 | 0.460 | 2.174 | −2.209 |
| optotype | 0.465 | 0.465 | 2.151 | |
| | 0.455 | 0.455 | 2.198 | |
| | 0.440 | 0.440 | 2.273 | |
| | 0.445 | 0.445 | 2.247 | |
| Vertical | 0.430 | 0.430 | 2.326 | −2.318 |
| optotype | 0.415 | 0.415 | 2.410 | |
| | 0.415 | 0.415 | 2.410 | |
| | 0.445 | 0.445 | 2.247 | |
| | 0.455 | 0.455 | 2.198 | |

When the optotype is horizontal, the mean dioptric power of the eye is −2.209.

When the optotype is vertical, the mean dioptric power of the left eye is −2.318.

The mean dioptric power D of the eye is D=[(−2.209)+(−2.318)]+2=−2.264.

For the testing of the dioptric power of simple hyperopic refractive error:

For the subject with hyperopia without clinically significant astigmatism, at 6 meters, if the visual acuity is greater than 1.0, then it is prompted that the subject has hyperopic refractive error. For the subject with the hyperopic refractive error, several convex lenses for adjusting the hyperopia (positive dioptric power) need to be provided for the subject.

In a case of hyperopia, the hyperopia spherical power, or astigmatism where there is difference between the hyperopia power on two axes, the test method for the subject with hyperopia with or without astigmatism is the same as that of the subject with myopia.

When the subject has hyperopia, three lenses with different positive dioptric powers are provided. One lens is +2 DS, one lens is +4 DS, and another lens is +6 DS.

The theoretical maximal hyperopic power combination is 6+4+2=+12 DS, (minimum is +2 DS), and the superposition combination of different lenses may be +2, +4, +6, +8, +10, and +12 DS, thus covering almost all common hyperopic power range.

For example, one person has hyperopia of +1 DS. Visually speaking, at 6 meters and beyond, the light emitted from an object is parallel when reaching the eye, assuming that the eye does not have a problem of refractive error, when these parallel lights enter the eye, they will be focused onto the retina, so hyperopia power cannot be obtained by applying without modifications the steps 2 to 36 in above methods which measure a myopia dioptric power with or without astigmatism. A conventional approach is to visit a glasses shop, a hospital, or an eye clinic to correct the hyperopia with convex lenses by an optometrist or a professional eye doctor. The correcting process aims at reaching neutralization to obtain the hyperopia dioptric power. Under or overcorrections happen during neutralization attempts. In case of overcorrection when the added lens exceeds the need of neutralizing the original hyperopia dioptric power, the eye acquire a myopia state with blurry vision and the added lens dioptric power needs to be reduced to reach neutralization.

For the subject with hyperopia without clinically significant astigmatism, at 6 meters, the optotype can clearly be seen, and the visual acuity of the subject is generally at 1.0 or above. A clinically myopia effect will be induced when the subject wears a convex +DS lenses that exceeds the hyperopia correction requirement. At this moment, the visual acuity of the subject is impaired, and at 6 meters, the visual acuity is lower than 1.0, and the optotype looks hazy.

Assuming that a subject with a dioptric power of +1 DS and the examiner do not know that the subject has a dioptric power of +1 DS, and a lens with a dioptric power of +2 DS is worn by the subject whom is theoretically over-corrected, which results in a state of −1 DS myopia effect to the subject. As long as the subject achieves the myopia effect, the above steps 2 to 36 in above methods for assessment and calculating the dioptric power of myopic refractive error is used to find the distance d when the subject sees the optotype clearly. The dioptric power is calculated by measuring the distance d, then the dioptric powers of the two lenses are summated, to get the original hyperopia dioptric power of the subject. Therefor the need of a large number of lenses for testing the subject on site and the need of a professional for performing optometry and correcting are avoided. By analogy assuming that the subject has the hyperopia of +3 DS and is provided with a lens of +2 DS, the subject can still see very clearly standing at a distance of 6 meters, indicating that the lens of +2 DS is insufficient. If this subject is provided with a lens of +4 DS, this time, an effect of −1 DS is achieved. The subject needs to go forward to see the optotype clearly. Again the actual dioptric power can be calculated by summation of dioptric powers after knowing the apparent dioptric power from measured distance d.

Similarly, if the subject has hyperopia of +5 DS, it can be calculated from distance d obtained by wearing a lens of +6 DS. If the hyperopia exceeds +6 DS, the three lenses are superposed to achieve +8 DS, +10 DS, +12 DS, and the like. This is enough to meet the range of most hyperopia. After all, very few people have the hyperopia above +6 DS clinically.

In step 3, if it is determined that the subject has clinically significant astigmatism in step 1 of FIG. 11, the required determination of the astigmatism axis and the calculation of the astigmatism power are specifically described below in combination with FIG. 9:

When the subject sees the optotype 32 at a distance of greater than or equal to 6 meters, the light from each same point of the optotype 32 is parallel when reaching the cornea. Assuming that the subject has simple astigmatism (0/−2 DC×180) without hyperopia or myopia, it means that in the horizontal direction the corneal curvature and the light focusing power are the weakest, and the spherical dioptric power is 0 DS. It also means in the vertical direction the corneal curvature and the light focusing power are the strongest, and the spherical dioptric power is −2 DS. On this occasion the light in the horizontal direction is focused to a rear focusing line which just overlaps the retina whereas the light in the vertical direction is focused on a front focusing line and lies in front of the retina.

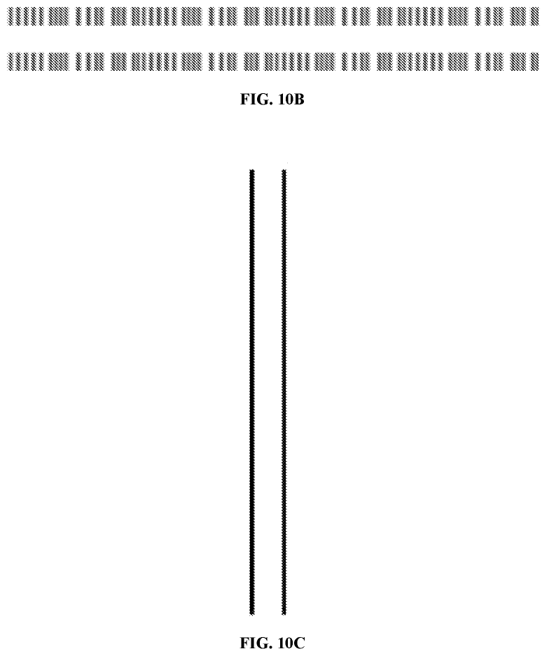

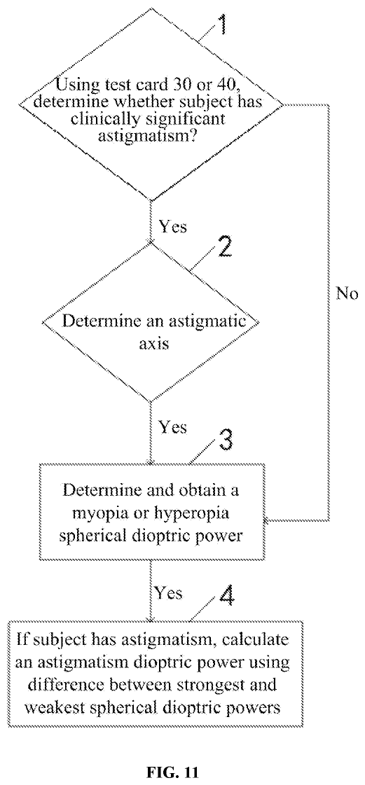

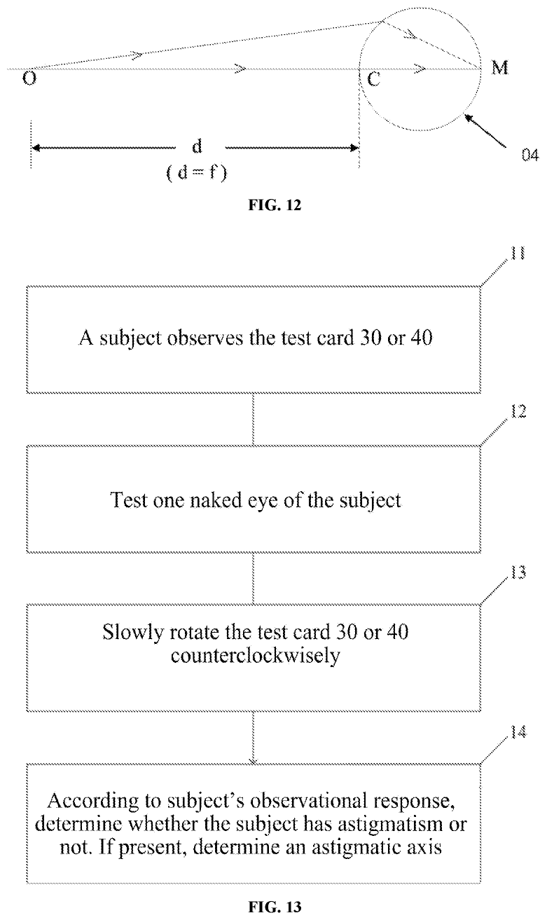

FIG. 10A, FIG. 10B, and FIG. 10C represent changes of the clarity of visual images of the front focusing line and the rear focusing line when the subject (0/−2 DC×180) observes two optotypes that are parallel in the horizontal direction or vertical direction.

The subject with myopic astigmatism has two different spherical dioptric powers, one is the weakest, and the other is the strongest. The weakest spherical dioptric power and the strongest spherical dioptric power respectively form its own focusing line. The distances of these two focusing lines relative to the retina are different. The focusing line that is the farthest from the center of the cornea is referred to as the rear focusing line, and the focusing line that is the closest to the center of the cornea is referred to as the front focusing line. The front focusing line and the rear focusing line also deviate from each other by ±90° on an axis.

Specific method steps for determining the astigmatism axis include step 11, step 12, step 13 and step 14.

In step 11, the subject observes the astigmatism test card 30 with the single white strip-shaped optotype 32 in the black background at a distance of 5 meters.

The test card adopts artificial illumination, for example, a direct illumination method; and the illumination is not lower than 300 lx, and the illumination is required to be uniform, constant, non-reflective, dazzle-free and should avoid direct sunlight or strong light illumination.

In step 12, one eye of the subject is tested first, and generally the right eye is tested first, and then the left eye is tested. When one eye is tested, the fellow eye is covered. The subject keeps the head upright and eyes looking straight ahead.

In step 13, a white strip-shaped astigmatism test card is rotated counterclockwisely and slowly.

This astigmatism test card is rotated counterclockwisely and slowly to allow the subject to see and decide in which axial direction the white strip looks relatively sharper/thinner. The rotating speed does not exceed 12.5°/second.

In step 14, according to the viewing response of the subject, whether the subject has astigmatism or not is determined and if astigmatism is present, the astigmatism axis is determined as well. When the subject with myopic astigmatism observes the single white strip-shaped astigmatism test card under the black background, its image is focused on two focusing lines which are in front of the retina, one is relatively closer to the retina, and the other is relatively less closer to the retina.

The image formed by the focusing line relatively closer to the retina is clearer than the image formed by the focusing line relatively less closer to the retina.

The axial angle at which the image formed by the closer rear focusing line appears to be relatively sharper/thinner as seen by the subject is recorded first. Then, this single white strip-shaped astigmatism test card is rotated more slowly clockwisely and counterclockwisely in repetition, so that the subject finds the axial angle in which the image is sharpest/thinnest, and the corresponding astigmatism axis is this axial angle 90°.

Furthermore, the steps to measure and calculate the astigmatism power, include step 31, step 32, step 33, step 34, step 35 and step 36.

In step 31, the subject observes the test card 50.

In step 32, in test card 50, the axis of the red strip-shaped optotype 52 is orientated to match the axis obtained from step 14 when the sharpest/thinnest image of the white strip-shaped optotype was observed by the subject with astigmatism;

In steps 33, 34, and 35, optically, the focusing line moves backward when the subject moves forward to get closer to the optotype 52. By making use of this phenomenon, the subject slowly moves from a distance of about 5 meters towards the optotype 52 and stops when the optotype 52 image becomes sharpest. At this moment, the distance from the eye of the subject to the optotype is measured, to calculate the weakest spherical dioptric power; this measured sharpest optotype distance d when a unilateral naked eye of the subject moves towards the optotype 52 is equivalent to the focal length f:

f=d (1)

The spherical dioptric power D is:

D=1/f (2)

Therefore, the first spherical dioptric power D₁ is obtained.

In step 35, then, the subject returns to a distance of 5 meters. The axial angle of the optotype 52 is rotated by 90° to be consistent with the axial angle of the astigmatism axis, and then the subject is asked to slowly move forward toward the optotype 52 and to stop when the optotype is sharpest. At this moment, the distance from the eye of the subject to the optotype 52 is measured again, and the strongest spherical dioptric power D₂ is obtained. (Note: at this moment, the rear focusing line moves behind the retina, which results in that an image of the optotype is hazy, so the subject only note a sharp image formed when the front focusing line overlaps the retina.)

In step 36, the rear focusing line is formed by the weakest spherical dioptric power, and the front focusing line is formed by the strongest spherical dioptric power.

The difference between the strongest spherical dioptric power and the weakest spherical dioptric power is the astigmatism dioptric power, therefore the astigmatism dioptric power can be calculated from the difference of the two dioptric powers (the weakest spherical dioptric power is subtracted from the strongest spherical dioptric power).

That is, the astigmatism dioptric power D′ of the eye of the subject is:

D′=D₂-D₁ (3)

In the presence of refractive error, when the spherical dioptric power is measured, firstly measure the weakest spherical dioptric power (which is a negative number in myopia) when the rear focusing line overlaps the retina. Secondly measure the strongest spherical dioptric power (which is a negative number in myopia) when the front focusing line overlaps the retina. Ten, the astigmatism dioptric power is obtained by subtracting the weakest spherical dioptric power from the strongest spherical dioptric power, and moreover, the astigmatism dioptric power is also negative. For example, (−5 DS)−(−3 DS)=−2 DC.

The advantage of the calculation is that each astigmatism dioptric power is represented by a negative number. A result obtained by subtracting a smaller negative number from a greater negative number is also a negative number. Thus avoiding confusion and error that the result is sometimes a positive number and sometimes a negative number.

Generally, it is acceptable clinically that the deviation of a measured astigmatism axis angle does not exceed 5° from the actual astigmatism axis angle. By the method of this present disclosure, such deviation does not exceed 2.5°.

The above steps are repeated to obtain the astigmatism dioptric power and axial angle of the fellow eye.

In combination of FIG. 15, steps for measuring and calculating the astigmatism dioptric power are as follows:

1. Base on the measured astigmatic axis, the astigmatism dioptric power is measured by means of a spherical dioptric power test card with a single red strip-shaped optotype in the black background. The axial angle of the red strip-shaped optotype is consistent with the measured astigmatic axis obtained when the above subject observes the sharpest/thinnest image of a rotating single white strip-shaped astigmatism test card. Measurement, recording, and calculation are conducted according to the method for calculating the spherical dioptric power described in step 24 and step 25, so that the weakest spherical dioptric power and the strongest spherical dioptric power are obtained. A simulation test needs to be conducted on the subject before a formal test.

2. In general, like other normal subjects, the subject being refracted always expresses some fluctuation in subjective accommodation status during optometric refraction. To mitigate the error induced by such fluctuation through one single measurement, the measurement is conducted five times in total. A mean value of the five measurements greatly reduces the deviation and thus achieves the clinically required accuracy, and the obtained mean value of f reflects the daily weakest spherical dioptric power of the subject.

3. Then, the red strip-shaped optotype is rotated by 90° to match the astigmatic axis (that is, in step 13 of an astigmatism axis measurement method, the axis at which the white strip-shaped optotype is sharpest/thinnest ±90°.) For example, the 15° obtained from the sharpest/thinnest white optotype is rotated to 105°, and so on. Measurement, recording, and calculation are performed according to the method for calculating the dioptric power in described step 26, so that the daily strongest spherical dioptric power of the subject is obtained.

4. The astigmatism dioptric power is solved according to the measured weakest spherical dioptric power and the strongest spherical dioptric power.

The astigmatism dioptric power of the subject may be obtained by subtracting the weakest dioptric power obtained from “step 34 of the astigmatism dioptric power measurement method” from the strongest spherical dioptric power obtained from “step 35 of the astigmatism dioptric power measurement method”, that is, subtracting the “dioptric power before rotating by 90°” from the “dioptric power after rotating by 90°”.

A calculation method for the astigmatism dioptric power is illustrated:

The following table data records the measured values of the distance d, and the calculation results of the dioptric power.

In a case of myopia, “−” needs to be added in front of the dioptric power.

Referring to the following table for illustration:

| | d(m) | f(m) | D = 1/f | Mean value of D |
|---|---|---|---|---|
| Before the | 0.275 | 0.275 | 3.636 | −3.734 |
| optotype is | 0.260 | 0.260 | 3.846 | |
| rotated by 90° | 0.275 | 0.275 | 3.636 | |
| | 0.270 | 0.270 | 3.704 | |
| | 0.260 | 0.260 | 3.846 | |
| After the | 0.255 | 0.255 | 3.922 | −4.160 |
| optotype is | 0.245 | 0.245 | 4.082 | |
| rotated by 90° | 0.245 | 0.245 | 4.082 | |
| | 0.220 | 0.220 | 4.545 | |
| | 0.240 | 0.240 | 4.167 | |

Before the optotype is rotated by 90°, the mean spherical dioptric power of the eye is −3.734 DS.

After the optotype is rotated by 90°, the mean spherical dioptric power of the eye is −4.160 DS.

The astigmatism dioptric power D of the eye is D=[(−4.160)−(−3.734)]=−0.426 DC.

For the subject with myopia and astigmatism:

When a certain subject has myopic dioptric power of −2 DS, astigmatic dioptric power of −3 DC and the astigmatic axis is 90° (which indicates that the weakest spherical dioptric power is −2 DS, the strongest spherical dioptric power is −5 DS, the difference between the two is −3 DC, and the astigmatic axis is at 90°). The rear focusing line of the subject is generated by the myopic dioptric power of −2 DS, and the front focusing line of the subject is generated by additional myopic astigmatic dioptric power of −3 DC. (In fact, because the subject has (−2)+(−3), the result is (−5 DS)). When the astigmatism axis of the subject is 90°, it represents that the light focusing power of the subject is on the line of 180°. It means that the focusing ability of the additional −3 DC is on the line of 180°.

For this subject, when the optotype and the rear focusing line are in the same axial angle, the myopia of −2 DS represents that a sharpest optotype image is formed when the subject is viewing 0.5 meter away from the optotype because the rear focusing line just overlaps the retina thus forming a sharpest optotype. Regarding the front focusing line, there is a 90° deviation relative to the optotype axial angle, thus not forming a sharp optotype image. Next, the optotype is rotated by 90°. At this moment, the axis of the optotype and the front focusing line are the same while there is a 90° deviation of axial angle between the rear focusing line and the optotype, thus not forming a sharp optotype image. When the subject moves forward to 0.2 meter position (the corresponding D is −5 DS), the front focusing line just overlaps the retina to form a sharpest optotype image.

For the subject with hyperopia and astigmatism:

For such subjects, the eye becomes myopic if a convex lens exceeding the hyperopia dioptric power of the subject is applied first. After that the hyperopia dioptric power, the astigmatism dioptric power, and the astigmatic axis of the subject can be obtained by applying the above myopia and astigmatism measurement steps and methods.

In conclusion, through the above operation, the hyperopia dioptric power, myopia dioptric power, astigmatism dioptric power, and the astigmatic axis can be obtained.

It is to be noted that, in the present disclosure, the refractive test card background color is not limited to the black, nor to the red or white optotypes. Color combinations with certain contrast, including blue+yellow, may also be adopted, so as to distinguish the optotype during testing.

Compared with conventional means, the test card and an optometry method using the test card of the present disclosure are convenient to operate, do not need complex devices and instruments, and are suitable for ordinary people to conduct self measurements at home. The method is easy to understand and master, and has high accuracy.

More importantly, the subject conducts the measurement in a most relaxed or near a most relaxed accommodation state without externally inserting a lens or replacing a lens. In this case, unnecessary interference and unwanted changes of accommodation or accommodation state are greatly reduced. By the method, an unnecessary dioptric power measurement error caused by subjective and empirical deviation of an optometrist is avoided.

Besides, the above method of the present disclosure can be implemented through application software. The software can be installed and used on a computer, a mobile phone, or a tablet computer. The required test content may be selected, and data is recorded and processed by operating directly on these interactive terminals. Displayed image content may be played on a computer display screen, or played by a television, or projected on a projection screen to play.

The application has two versions, an online APP and an offline APP. In an online APP version, main core data is stored in a cloud server. Only a small amount of simple data is downloaded to an interactive device terminal (iPad and the like) for users to select the device to install. Data processing is performed through communication connection with the background server. In an offline APP version, all data is directly downloaded to the interactive device, and then the device is bonded for using.

In the present disclosure an optometry for refractive error (including the hyperopia dioptric power, myopia dioptric power, astigmatism dioptric power, and the astigmatic axis) is originally provided, which is simple, objective, and convenient to use, and meanwhile, simple and clear optotypes are also originally provided.

The simple and clear optotype is that:

Whether there is astigmatism and an astigmatism axis are measured by using a single white strip-shaped astigmatism test card (not limited to other combinations of colors and shapes) under the black background. The optometry of refractive error is conducted by using the test card, so that the astigmatic axis accurate to ±2.5° can be found accurately, which is more accurate compared with ±5° achieved by conventional methods.

The spherical dioptric power is measured by using a single red strip-shaped optotype test card (not limited to other combinations of colors and shapes) under the black background. The spherical dioptric power and the astigmatism dioptric power can be calculated by using the test card to conduct an optometry for refractive error.

The basic concepts have been described above. It is apparent to those skilled in the art that the above present disclosures are merely examples and not intended to limit this application. Those skilled in the art may make various modifications, improvements, and corrections to this application, even though not specified herein. Such modifications, improvements, and corrections are suggested in this application, so such modifications, improvements, and corrections still fall within the spirit and scope of the exemplary embodiments of this application.

In addition, specific terms are used in this application to describe the embodiments of this application. For example, “one embodiment”, “an embodiment”, and/or “some embodiments” mean/means a certain feature, structure, or characteristic related to at least one embodiment of this application. Therefore, it is to be emphasized and noted that “an embodiment”, or “one embodiment”, or “an alternative embodiment” mentioned twice or more at different positions in the description does not always refer to the same embodiment. In addition, some features, structures, or characteristics in one or more embodiments of this application may be appropriately combined.

Some aspects of this application can be executed completely by hardware, or software (including firmware, resident software, microcodes, etc.), or by a combination of the hardware and the software. The hardware or software may be called a “data block”, “module”, “engine”, “unit”, “component”, or “system”. The processor may be one or more Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors or combinations thereof. In addition, each aspect of this application may be represented as a computer product in one or more computer-readable media, and the product includes computer-readable program codes. For example, the computer-readable medium may include, but is not limited to, a magnetic storage device (such as a hard disc, a floppy disc, or a magnetic tape), a compact disc (such as a Compressed Disc (CD), a Digital Video Disc (DVD)), a smart card, and a flash device (such as a card, a stick, or a key drive).

The computer-readable medium may include a propagation data signal with computer program code therein, for example, on a baseband or as a part of a carrier. The propagated signal may be represented in many forms, including an electromagnetic form, an optical form, and the like, or a proper combination form. The computer-readable medium may be any computer-readable medium except a computer-readable storage medium, and the medium is connected to at least one instruction execution system, apparatus, or device to implement a program for communication, propagation, or transmission. The program code in the computer-readable storage medium may be propagated through any suitable medium, including radio, a cable, an optical fiber, a Radio Frequency (RF) signal, a similar medium, or any combination of the above media.

The basic concepts have been described above. It is apparent to those skilled in the art that the above present disclosures are merely examples and not intended to limit this application. Those skilled in the art may make various modifications, improvements, and corrections to this application, even though not specified herein. Such modifications, improvements, and corrections are suggested in this application, so such modifications, improvements, and corrections still fall within the spirit and scope of the exemplary embodiments of this application.

In addition, specific terms are used in this application to describe the embodiments of this application. For example, “one embodiment”, “an embodiment”, and/or “some embodiments” mean/means a certain feature, structure, or characteristic related to at least one embodiment of this application. Therefore, it is to be emphasized and noted that “an embodiment”, or “one embodiment”, or “an alternative embodiment” mentioned twice or more at different positions in the description does not always refer to the same embodiment. In addition, some features, structures, or characteristics in one or more embodiments of this application may be combined as appropriate.

Similarly, it is to be noted that, for simplifying the expressions disclosed in this application to help to understand one or more embodiments of the disclosure, multiple features may sometimes be incorporated into one embodiment, drawing, or the description thereof in the above description about the embodiments of this application. However, such a disclosure method does not mean that an object of this application needs more features than those mentioned in the claims. In practice, the features of the embodiment are fewer than all features of a single embodiment disclosed above.

Numerals describing the numbers of components and attributes are used in some embodiments. It is to be understood that such numerals for describing the embodiments are modified with modifiers “about”, “approximately”, or “substantially” in some examples. Unless otherwise specified, “about”, “approximately”, or “substantially” represents that the numeral allows a change of ±20%. Correspondingly, in some embodiments, numerical parameters used in the description and the claims are all approximate values, and the approximate values may change according to characteristics required by individual embodiments. In some embodiments, the numerical parameter should consider specified valid digits and adopt a general digit retention method. Although numerical ranges and parameters, in some embodiments of this application, used to confirm the breadths of scopes thereof are approximate values, such numerical values are set as accurately as possible in a possible scope in specific embodiments.

Although this application has been described with reference to the current specific embodiments, those of ordinary skill in the art should recognize that the above embodiments are merely used to describe this application, various equivalent changes or replacements may also be made without departing from the spirit of this application. Therefore, as long as the changes and modifications of the above embodiments are within the scope of substantive spirit of this application, they all fall within the scope of the claims of this application.

What is claimed is:

1. A refractive test card, comprising an optotype under a black background, wherein the optotype comprises a partition unit at a central position, and the partition unit visually separates the optotype.

2. The refractive test card according to claim 1, wherein the optotype comprises a strip-shaped optotype, and color of the strip-shaped optotype comprises either of white and red.

3. The refractive test card according to claim 2, wherein the optotype comprises a cross-shaped optotype, and color of the cross-shaped optotype comprises white.

4. The refractive test card according to claim 3, wherein a shape of the partition unit comprises a rectangle or a circle.

5. The refractive test card according to claim 4, wherein the optotype has a length of 260 mm±50 mm, and a width of 5 mm±2 mm.

6. The refractive test card according to claim 5, wherein the partition unit has a width of less than or equal to 5 mm, and a height range of 5 mm±2 mm.

7. The refractive test card according to claim 6, wherein the refractive test card comprises an astigmatism test card and a spherical dioptric power test card; wherein the astigmatism test card comprises a white strip-shaped optotype and a cross-shaped optotype, and the spherical dioptric power test card comprises a red strip-shaped optotype.

8. A measurement method for implementing the refractive test card according to claim 1, comprising:
step a, determining whether a subject has astigmatism based on sharpness of contrast between an optotype and a black background of an astigmatism test card observed by the subject;
step b, in response to determining that the subject has astigmatism, determining an astigmatic axis; and in response to determining that the subject does not have astigmatism, turning to step d;
step c, determining whether the subject has myopia or hyperopia based on sharpness of contrast between an optotype and a black background of a spherical dioptric power test card observed by the subject;
step d, testing, measuring and calculating a spherical dioptric power of the subject; and
step e, testing, measuring and calculating an astigmatism dioptric power of the subject in a case that the subject has astigmatism.

9. The measurement method according to claim 8, wherein determining the astigmatic axis in step b comprises:
step b1, testing an unilateral naked eye of the subject by rotating the astigmatism test card at a speed no higher than 12.5°/second, to obtain a maximum sharpness axis of the astigmatism test card observed by the subject; and
step b2, determining the maximum sharpness axis±90° as the astigmatic axis.

10. The measurement method according to claim 9, wherein step d further comprises:
step d1, obtaining a sharpest image distance d and a focal length f by moving the unilateral naked eye of the subject towards the spherical dioptric power test card:
f=d (1);
step d2, obtaining a first spherical dioptric power D₁ according to spherical dioptric power D:
D=1/f (2);
step d3, rotating the spherical dioptric power test card by 90°, and repeating steps d1 and d2 to obtain a second spherical dioptric power D₂;
step d4, calculating the spherical dioptric power D of an eye of the subject:
D=(D₁+D₂)/2 (3); and
step d5, repeating steps d1˜d4 to obtain a spherical dioptric power of a fellow eye.

11. The measurement method according to claim 10, wherein step e further comprises:
step e1, placing the spherical dioptric power test card in the sharpest axis of step b1;
step e2, obtaining the sharpest image distance d and the focal length f by moving the unilateral naked eye of the subject towards the spherical dioptric power test card:
f=d (1);
step e3, obtaining the first spherical dioptric power D₁ according to the spherical dioptric power D:
D=1/f (2);
step e4, placing the spherical dioptric power test card in the astigmatic axis, and repeating steps e1˜e3 to obtain a second spherical dioptric power D₂;
step e5, calculating the astigmatism dioptric power D′ of the eye of the subject:
D′=D₂−D₁ (3);and
step e6, repeating steps e1˜e5 to obtain the astigmatism dioptric power of the fellow eye.

12. The measurement method according to claim 8, in step a:
rotating the astigmatism test card; and in a case that the subject observes that there is no change in the sharpness of contrast between the optotype and the black background, determining that the subject does not have astigmatism; and otherwise, determining that the subject has astigmatism.

13. The measurement method according to claim 9, in step a:
rotating the astigmatism test card; and in a case that the subject observes that there is no change in the sharpness of contrast between the optotype and the black background, determining that the subject does not have astigmatism; and otherwise, determining that the subject has astigmatism.

14. A measurement method for implementing the refractive test card according to claim 2, comprising:
step a, determining whether a subject has astigmatism based on sharpness of contrast between an optotype and a black background of an astigmatism test card observed by the subject;
step b, in response to determining that the subject has astigmatism, determining an astigmatic axis; and in response to determining that the subject does not have astigmatism, turning to step d;
step c, determining whether the subject has myopia or hyperopia based on sharpness of contrast between an optotype and a black background of a spherical dioptric power test card observed by the subject;
step d, testing, measuring and calculating a spherical dioptric power of the subject; and
step e, testing, measuring and calculating an astigmatism dioptric power of the subject in a case that the subject has astigmatism.

15. The measurement method according to claim 14, wherein determining the astigmatic axis in step b comprises:
step b1, testing an unilateral naked eye of the subject by rotating the astigmatism test card at a speed no higher than 12.5°/second, to obtain a maximum sharpness axis of the astigmatism test card observed by the subject; and
step b2, determining the maximum sharpness axis±90° as the astigmatic axis.

16. The measurement method according to claim 15, wherein step d further comprises:
step d1, obtaining a sharpest image distance d and a focal length f by moving the unilateral naked eye of the subject towards the spherical dioptric power test card:
f=d (1);
step d2, obtaining a first spherical dioptric power D₁ according to spherical dioptric power D:
D=1/f (2);
step d3, rotating the spherical dioptric power test card by 90°, and repeating steps d1 and d2 to obtain a second spherical dioptric power D₂;
step d4, calculating the spherical dioptric power D of an eye of the subject:
D=(D₁+D₂)/2 (3); and
step d5, repeating steps d1˜d4 to obtain a spherical dioptric power of a fellow eye.

17. The measurement method according to claim 16, wherein step e further comprises:
step e1, placing the spherical dioptric power test card in the sharpest axis of step b1;
step e2, obtaining the sharpest image distance d and the focal length f by moving the unilateral naked eye of the subject towards the spherical dioptric power test card:
f=d (1);
step e3, obtaining the first spherical dioptric power D₁ according to the spherical dioptric power D:
D=1/f (2);
step e4, placing the spherical dioptric power test card in the astigmatic axis, and repeating steps e1˜e3 to obtain a second spherical dioptric power D₂;
step e5, calculating the astigmatism dioptric power D′ of the eye of the subject:
D′=D₂−D₁ (3); and
step e6, repeating steps e1˜e5 to obtain the astigmatism dioptric power of the fellow eye.

18. The measurement method according to claim 14, in step a:
rotating the astigmatism test card; and in a case that the subject observes that there is no change in the sharpness of contrast between the optotype and the black background, determining that the subject does not have astigmatism; and otherwise, determining that the subject has astigmatism.

19. The measurement method according to claim 15, in step a:
rotating the astigmatism test card; and in a case that the subject observes that there is no change in the sharpness of contrast between the optotype and the black background, determining that the subject does not have astigmatism; and otherwise, determining that the subject has astigmatism.
